(19) United States
(12) Patent Application Publication
(10) Pub. No.: US 2025/0027204 A1
(43) Pub. Date: Jan. 23, 2025

(54) Spray Pyrolysis of Li-Salt Films

(71) Applicant: Massachusetts Institute of Technology, Cambridge, MA (US)

(72) Inventors: Jennifer Lilia Rupp, Starnberg (DE); Yuntong Zhu, Kirkland, WA (US); Lingping Kong, Cambridge, MA (US); Moran Balaish, Somerville, MA (US)

(21) Appl. No.: 18/710,382

(22) PCT Filed: Nov. 15, 2021
(86) PCT No.: PCT/US2021/059339

§ 371 Date: May 15, 2024

(51) Int. Cl. C23C 18/12; G01N 33/00

(52) U.S. Cl. CPC C23C 18/1258; C23C 18/1291; C23C 18/1295; G01N 33/0042

(57) ABSTRACT

A method for making a lithium salt film includes heating a substrate, spraying a mixture with a spray nozzle onto the substrate to form a precursor film, and annealing the precursor film to form the lithium salt film. The lithium salt film has a thickness of about 400 nm to about 100 pm. The spray mixture includes a first precursor comprising a lithium ion, a second precursor comprising an anion, and a solvent.

### BACKGROUND

Lithium salt thin films have been deposited using several physical vapor deposition (PVD) processes. PVD processes, including RF and DC sputtering, pulsed laser deposition, and e-beam and thermal evaporation processes, use vacuum techniques to deposit thin films by transporting material from a condensed matter source via the gas phase to a substrate, where the thin film is formed. Many PVD processes involve ballistic transport of material, where material is transferred from the source to the substrate with very few collisions with other particles. The material transferred from the condensed phase to the gas phase obtains energy from the condensed matter source, and this energy is largely preserved until the material hits the substrate surface. To preserve ballistic transport, deposition is conducted under vacuum, at a pressure conventionally less than about 75 mTorr.

Radio frequency (RF) sputtering is a type of PVD that has been used to deposit amorphous thin films of lithium salts like Li₃PO₄ and Li₂SO₄. Sputter deposition deposits a thin film by ejecting material from a target condensed matter source onto a substrate. Conventionally, material is ejected from the target using an energized argon plasma. Sputtering chambers use strong electric and magnetic fields to confine charged plasma particles close to the surface of the sputter target. In RF sputtering, an RF power supply is used to vary the sign of the anode-cathode bias at a high rate to deter charge build up on an insulating target.

There are several limitations to PVD of lithium salt films. Since deposition rates are low (e.g., about 100 nm per hour), this technique is not well-suited for depositing thicker films (e.g., >1 μm). PVD is conventionally used to deposit films on small areas (e.g., less than 1 cm²), and is not well-suited for depositing films over larger areas. It can be difficult to control film thickness and morphology using PVD processes. It can also be difficult to control the stoichiometric composition of the film. Light elements like lithium deflect more easily than heavier elements, which can result in composition variation in the deposited film.

Furthermore, there are several disadvantages to using vacuum conditions, which are used for PVD processes. Deposition processes performed under vacuum conditions are often expensive. They often use specialized reaction chambers with expensive vacuum pumps. PVD processes conventionally use high-purity gases, which can also be costly. Vacuum-based processes can also be difficult to scale-up, since maintaining vacuum conditions in larger chambers presents an engineering challenge. Vacuum-based processes also tend to have longer preparation and processing times. Pumping down the reaction chamber to the low pressure used for processing can take a significant period of time. Purging the reaction chamber between processing steps can also be time consuming. Therefore, processes that use vacuum conditions are often economically unattractive.

### SUMMARY

As described herein, alkali metal salts (e.g., lithium salts), alkaline earth metal salts, and/or composite metal salts can be deposited using spray pyrolysis onto a substrate (e.g., a metal, a metal oxide, a metal nitride, a metal alloy, a borosilicate, carbon, silicon, silicon dioxide, or a polyimide). One embodiment includes a method of making a metal salt film. The method includes heating a substrate and, while heating the substrate, spraying a mixture onto the substrate to form a precursor film (e.g., at a spray rate of about 5 mL per hour to about 10 mL per hour). The spray mixture includes a first precursor including at least one alkali metal ion (e.g., Li) or alkaline earth metal ion, a second precursor including an anion, and a solvent. After the precursor film is deposited, it is annealed to form the metal salt film (e.g., with the metal salt film having a temperature of about 400° C. for about 10 minutes to about 30 minutes). The metal salt film has a thickness of about 400 nm to about 100 μm, or about 600 nm to about 70 μm, or about 800 nm to about 40 μm, or about 1 μm to about 10 μm. The metal salt film comprises the alkali metal ion and/or alkaline earth metal ion and the anion from the spray mixture. During the step of annealing, the precursor film has a temperature of about 100° C. to about 800° C. In an example, during the spraying step, the substrate may be heated with a heating element thermally coupled to the substrate. The heating element may have a temperature of 100° C. to 400° C. The solvent may include at least one of water, an alcohol, an ester, a carbonate, or a ketone.

The metal salt film may include a lithium salt film, where an alkali metal ion dissolved in the solvent in the spray mixture is a lithium ion. In this example, the concentration of lithium ion in the solvent has a stoichiometric excess (e.g., about 5% to about 300%) greater than the stoichiometric amount of the lithium ion in the resulting lithium salt film. The first precursor may include lithium acetate, lithium nitrate, lithium hydroxide, and/or lithium azide. The second precursor may include at least one of a sulfate ion, a nitrate ion, a nitride ion, a phosphate ion, a fluoride ion, a chloride ion, a bromide ion, a perchlorate ion, or an azide ion (e.g., ammonium sulfate). The lithium salt film may include lithium nitrate, lithium nitride, lithium sulfate, lithium phosphate, lithium fluoride, lithium chloride, lithium bromide, lithium hydroxide, lithium perchlorate, and/or lithium azide.

The spray mixture may further comprise a third precursor comprising a second metal ion so that the metal salt film includes the second metal ion (e.g., as a composite including a first metal salt and a second metal salt). The second metal ion may include at least one of zinc, calcium, potassium, sodium, bismuth, cerium, zirconium, iron, yttrium, lanthanum, tantalum, or beryllium. For example, the first precursor may include lithium sulfate and the third precursor may include calcium sulfate. The lithium sulfate and calcium sulfate may be in approximately a one-to-one ratio.

As an example, the steps of spraying and heating may be performed in a substantially inert gas environment. In this example, the surface of the metal salt film may be coated with a capping layer to prevent or substantially mitigate atmospheric contamination of the metal salt film.

As an example, the metal salt film may be a sensing electrode in an electrochemical gas sensor device (e.g., a sulfur dioxide gas sensor). As another example, the metal salt film may be used in a solid-state battery as part of a solid-state electrolyte.

Another embodiment is a lithium salt film deposition system. The system includes a spray pyrolysis apparatus and a post-annealing apparatus. The spray pyrolysis apparatus includes a precursor solution including lithium acetate and ammonium sulfate, a spray nozzle to create a spray mist of the precursor solution, a substrate positioned to be contacted by at least a portion of the spray mist, and a first heating source configured to heat the substrate (e.g., so that the substrate has a temperature of about 100° C. to about 400° C.). The post-annealing apparatus includes a chamber and a second heating source thermally coupled to the chamber and configured to heat the substrate so that the substrate has a temperature of about 100° C. to about 800° C. The post-annealing apparatus may be a tube furnace. The tube furnace may include an inert gas source fluidically coupled to the chamber of the tube furnace. All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows a spray pyrolysis system.

FIG. 1B shows an annealing chamber to anneal the as-deposited metal salt layer deposited with the system in FIG. 1A.

FIG. 1C shows part of a spray pyrolysis system enclosed in an inert environment.

FIG. 1D shows a lithium salt layer with a capping layer deposited with the system in FIG. 1C.

FIG. 2 shows a cross section of a section of a solid-state battery using a lithium salt solid electrolyte deposited via a spray pyrolysis system.

FIG. 3 shows a gas sensor using a lithium salt sensing electrode deposited via a spray pyrolysis system.

FIG. 4A is a graph showing thermogravimetric and calorimetric changes in lithium acetate with increasing temperature.

FIG. 4B is a graph showing thermogravimetric and calorimetric changes in ammonium sulfate with increasing temperature.

FIG. 4C is a graph showing thermogravimetric and calorimetric changes in calcium acetate with increasing temperature.

FIG. 5A shows an as-deposited lithium sulfate film deposited at a spray rate of 5 mL per hour at 200° C.

FIG. 5B shows a lithium sulfate film annealed at 400° C. deposited at a spray rate of 5 mL per hour.

FIG. 5C shows a lithium sulfate film annealed at 500° C. deposited at a spray rate of 5 mL per hour.

FIG. 5D shows a lithium sulfate film annealed at 700° C. deposited at a spray rate of 5 mL per hour.

FIG. 6A shows an as-deposited lithium sulfate film deposited at a spray rate of 10 mL per hour at 200° C.

FIG. 6B shows a lithium sulfate film annealed at 500° C. deposited at a spray rate of 10 mL per hour.

FIG. 6C shows a lithium sulfate film annealed at 700° C. deposited at a spray rate of 10 mL per hour.

FIG. 7A shows Raman spectra of lithium sulfate films deposited at a spray rate of 5 mL per hour annealed at different temperatures.

FIG. 7B shows Raman spectra of lithium sulfate films deposited at a spray rate of 10 mL per hour annealed at different temperatures.

FIG. 8A shows a lithium sulfate film annealed at 400° C. for 60 minutes with a heating rate of 10° C. per minute.

FIG. 8B shows a lithium sulfate film annealed at 400° C. for 60 minutes with a heating rate of 20° C. per minute.

FIG. 9 shows Raman spectra of lithium sulfate films annealed at 400° C.

FIG. 10A shows an as-deposited composite film.

FIG. 10B shows a composite film annealed at 400° C.

FIG. 10C shows a composite film annealed at 500° C.

FIG. 10D shows a composite film annealed at 700° C.

FIG. 11 shows Raman spectra of composite films annealed at different temperatures.

FIG. 12 shows a Raman spectrum of a lithium perchlorate film.

### DETAILED DESCRIPTION

Described herein are systems and methods for forming amorphous and/or crystalline metal salt films using wet-chemical spray pyrolysis. The methods described herein are widely applicable to depositing a wide variety of metal salt chemistries, including alkali metal salts, alkaline earth metal salts, and composite salts. For example, these methods can deposit lithium salt films, calcium salt films, and composite films including lithium salt and calcium salt. The thickness of the metal salt films can be tuned between 300 nm and 100 μm. The surface roughness, phase, and crystallinity can be modified by tuning spray and/or post-annealing parameters and conditions. These systems and methods provide control of composition, film thickness and surface morphology.

Compared to other deposition techniques, the spray pyrolysis systems and methods described herein provide several advantages. RF sputtering under vacuum has been used to produce lithium phosphorous sulfuric oxynitrides (LiPSON) thin films, but these films conventionally had a thickness less than 1 μm. (While RF sputtering could be used to deposit a film with a thickness greater than 1 μm, its low growth rate makes it impractical to do so.) Spray pyrolysis has been used to produce composite films of lithium sulfate and calcium sulfate using a direct spray of a colloidal solution of lithium sulfate and calcium sulfate. For example, in U.S. Provisional Appl. Ser. No. 63/154,336, filed Feb. 26, 2021, which is incorporated herein by reference in its entirety, lithium sulfate films were deposited using this direct spray method. Film thickness using this direct spray method was limited to greater than 10 μm. This direct spray method also had poor control of thickness and morphology. Elevated temperatures (greater than 700° C.) were used to post-anneal the films to densify and develop adhesion with other functional layers.

In comparison to these other techniques, the systems and methods described herein provide lithium salt film thicknesses that were not previously possible, in the range of 1 μm to 10 μm. These methods can be used to deposit films with thicknesses of 300 nm to 100 μm. These methods are well-suited for depositing lithium salt films in this thickness range because of their reasonable growth rates (e.g., about 50 nm per hour to about 10 μm per hour). Another advantage is that these systems and methods use reduced processing temperatures compared to previous techniques and do not require vacuum conditions, making them economically suitable for scalable production.

These systems and methods also expand the variety of lithium salt thin films that can be deposited. A large variety of salt films can be deposited via these systems and methods. Salt films that can be deposited include salts with alkali metals (lithium, sodium, potassium, rubidium, and/or cesium) and/or alkali earth metals (beryllium, magnesium, calcium, strontium, and/or barium). Salt films can also have a variety of anions, including sulfate, chloride, perchlorate, fluoride, bromide, nitride, nitrate, arsenic hexafluoride, hexafluorophosphate, triflate, bistriflimide (TFSI), and/or bis(oxalato)borate (BOB). For example, salt films include lithium sulfate, lithium chloride, lithium perchlorate, lithium fluoride, lithium bromide, lithium nitrate, lithium nitride, and lithium sulfide.

The spray pyrolysis method described herein is a chemical-solution based process for depositing metal salt thin films, including lithium salt thin films. This technique forms a thin film by spraying a precursor solution onto a heated substrate. The precursor solution is directed at the heated substrate as a fine mist or aerosol using a nozzle. The deposited film may be heat treated using post-annealing following deposition.

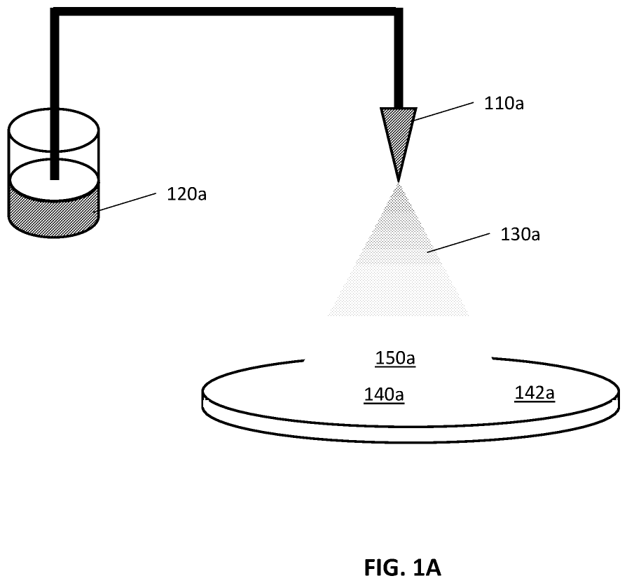

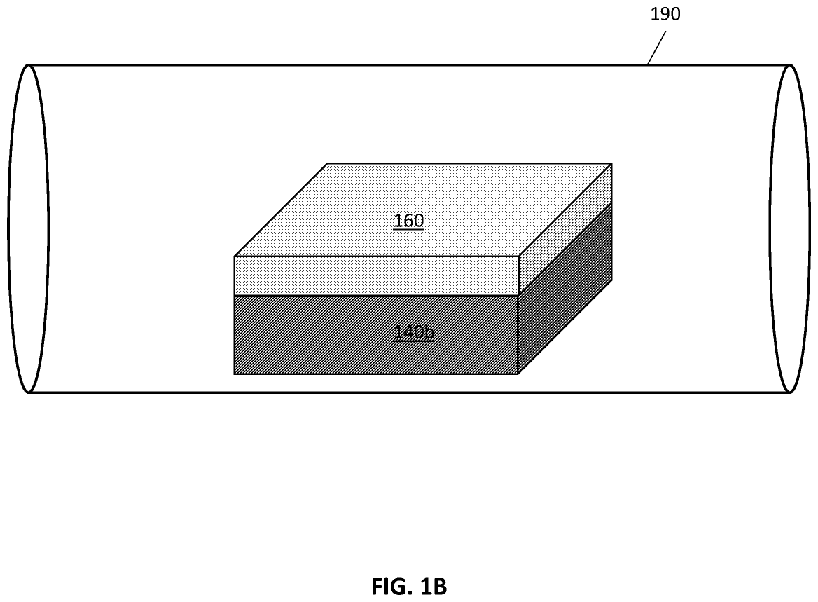

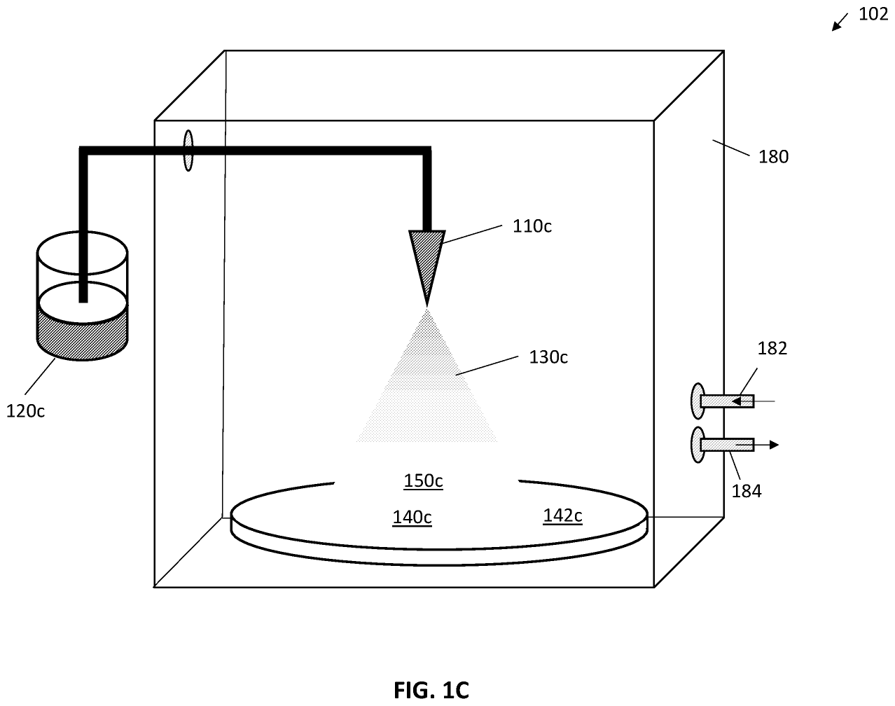

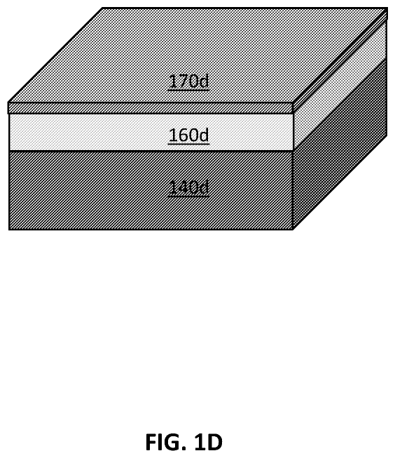

FIG. 1A shows a spray pyrolysis system 100. The system 100 includes a spray nozzle 110a (e.g., an atomizer) that is fluidically coupled to a precursor solution 120a. The spray nozzle 110a produces a spray mist 130a. The spray mist 130a includes droplets of precursor solution 120a. The spray mist 130a is directed towards a substrate 140a, where the thin film is formed. The substrate 140a is thermally coupled to a heating element 142a that heats the substrate 140a. After deposition, the thin film 150a may be heat treated (i.e., post-annealed) at an elevated temperature to form a post-annealed thin film 160, shown in FIG. 1B.

The precursor solution 120a includes at least two precursors, where at least one of precursors is an alkali metal precursor (e.g., a lithium precursor) or alkali earth metal precursor (e.g., a calcium precursor) and at least one of the precursors is an anion precursor. The precursors may decompose at elevated temperatures to form the desired alkali metal salt or alkali earth metal salt (e.g., lithium salt or calcium salt) or composite salt film (e.g., lithium salt and calcium salt).

The precursors are dissolved in the solvent. The boiling point of the solvent is a factor used in choosing the solvent. The solvent may be a mixture of more than one solvent to tune the boiling point. The boiling point of the solvent affects the resulting film morphology, phase, and level of impurities. The solvent may include a substituted or unsubstituted C₁₋₂₀ alcohol, a substituted or unsubstituted C₁₋₄₀ ester, a substituted or unsubstituted C₂₋₂₀ carbonate, a substituted or unsubstituted C₁₋₂₀ ketone, water, or a combination thereof. In one embodiment, the solvent is water. In another embodiment, the solvent is water, methanol, a substituted propanol, e.g., 1-methoxy-2-propanol, or a combination thereof. The boiling point of the solvent is about 15° C. to about 450° C. Preferably, the boiling point of the solvent is about 50° C. to about 400° C. In one embodiment, the boiling point of the solvent is about 80° C. to about 120° C. (e.g., 80° C., 90° C., 100° C., 110° C., or 120° C.). In another embodiment, the boiling point of the solvent is about 250° C. to about 400° C. (e.g., 250° C., 300° C., 350° C., or 400° C.).

The lithium precursor is present in the precursor solution 120a. It is a compound that can dissolve in the precursor solvent. The content of the lithium precursor in the solution may be a stoichiometric excess greater than the stoichiometric amount of the lithium ion in the resulting lithium salt film. The stoichiometric excess may be about 5% to about 300% (e.g., about 5%, 10%, 20%, 30%, 40%, 50%, 60%, 70%, 80%, 90%, 100%, 150%, 200%, or 300%). Preferably, the stoichiometric excess is about 50%. This stoichiometric excess is intended to counteract lithium evaporation during deposition, which may otherwise result in a thin film that is lithium poor. The concentration of the lithium precursor in the solution may be about 0.001 molar (M) to about 1 M. Preferably, the lithium precursor concentration may be about 0.005 M to about 0.05 M. The lithium precursor may include lithium acetate, lithium nitrate, lithium nitride, lithium hydroxide, lithium azide, lithium oxide, lithium carbonate or a combination thereof.

The anion precursor is also present in the precursor solution 120a. It is also a compound that can dissolve in the precursor solvent. The content of the anion precursor in the solution may be within about ±10% of the stoichiometric amount of the anion in the resulting lithium salt film. The concentration of the anion precursor in the solution may be about 0.001 molar (M) to about 1 M. Preferably, the anion precursor concentration may be about 0.005 M to about 0.05 M (e.g., 0.005 M, 0.01 M, 0.02M, 0.03 M, 0.04 M, or 0.05 M). The anion in the anion precursor may include a sulfate, carbonate, nitrate, sulfide, perchlorate, halide, nitride, arsenic hexafluoride, hexafluorophosphate, triflate, bistriflimide (TFSI), and/or bis(oxalato)borate (BOB), The cation in the anion precursor may be ammonium, tetramethylammonium, phenelzine, hydroxylamine, hydroxylammonium, or a combination thereof. Preferably, the cation is ammonium because ammonium is evolved during precursor film annealing, leaving the anion freely available for the formation of the metal salt of interest with relatively low impurity levels. Examples of anion precursors include ammonium sulfate, ammonium carbonate, ammonium nitrate, ammonium sulfide, ammonium chlorate, ammonium halide or a combination thereof. Two or more anion precursors may be used to form a thin film that includes a mixture of salts.

The precursor solution 120a may include one or more additional precursors to form a thin film composite film. Additional precursors include calcium precursors, zinc precursors, potassium precursors, sodium precursors, bismuth precursors, cerium precursors, zirconium precursors, iron precursors, yttrium precursors, lanthanum precursors, tantalum precursors, and/or barium precursors. The calcium precursor may be calcium acetate, calcium nitrate, calcium nitride, calcium hydroxide, calcium azide, or a combination thereof. The zinc precursor may be zinc acetate, zinc nitrate, zinc nitride, zinc hydroxide, zinc azide or a combination thereof. The potassium precursor may be potassium acetate, potassium nitrate, potassium nitride, potassium hydroxide, potassium azide or a combination thereof. The sodium precursor may be sodium acetate, sodium nitrate, sodium nitride, sodium hydroxide, sodium azide, or a combination thereof. The bismuth precursor may be bismuth acetate, bismuth nitrate, bismuth nitride, bismuth hydroxide, bismuth azide, or a combination thereof. The cerium precursor may be cerium acetate, cerium nitrate, cerium nitride, cerium hydroxide, cerium azide or a combination thereof. The zirconium precursor may be zirconium acetate, zirconium nitrate, zirconium nitride, zirconium hydroxide, zirconium azide, or a combination thereof. The iron precursor may be iron acetate, iron nitrate, iron nitride, iron hydroxide, iron azide, or a combination thereof. The yttrium precursor may be yttrium acetate, yttrium nitrate, yttrium nitride, yttrium hydroxide, yttrium azide, or a combination thereof. The lanthanum precursor may be lanthanum acetate, lanthanum nitrate, lanthanum nitride, lanthanum hydroxide, lanthanum azide, or a combination thereof. The tantalum precursor may be tantalum acetate, tantalum nitrate, tantalum nitride, tantalum hydroxide, tantalum azide, or a combination thereof. The barium precursor may be barium acetate, barium nitrate, barium nitride, barium hydroxide, barium azide, or a combination thereof. The concentration of any of these precursors in the solution may be about 0.001 molar (M) to about 1 M.

The precursor solution 120a is sprayed onto the heated substrate 140a. The precursor solution 120a is sprayed with a pressure of about 0.1 bar to about 2 bar. Preferably, the precursor solution 120a is sprayed with a pressure of about 0.2 bar to about 0.2 bar. The precursor solution 120a may be sprayed using a carrier gas. The carrier gas may be compressed air, argon, nitrogen, oxygen, or a combination thereof. The precursor solution 120a may be sprayed on the heated substrate 140a at a rate of about 1 milliliter per hour (mL/hr) to about 50 mL/hr (e.g., 1 mL/hr, 2 mL/hr, 3 mL/hr, 4 mL/hr, 5 mL/hr, 10 mL/hr, 20 mL/hr, 40 mL/hr or 50 mL/hr). Preferably, the precursor solution 120 is sprayed onto the heated substrate 140a at a rate of about 5 mL/hr to about 10 mL/hr. The amount of material sprayed onto the substrate 140a is chosen based on the desired thickness of the lithium salt thin film and the size of the substrate. The thickness of the film increases with increasing volume of precursor solution 120a sprayed onto the substrate 140a.

The substrate 140a is heated by the heating element 142a. Without being bound by any theory, heating the substrate may evaporate or decompose the solvent in the precursor solution 120a before or after the precursor solution 120a contacts the surface of the substrate (e.g., while in the form of a spray mist 130a or after contacting the substrate). The heating element may be a convection heater, an infrared heater, a resistive heater, or a combination thereof. Alternatively, the substrate may be heated via a light source (e.g., a laser) emitting light in a wavelength range (e.g., infrared light). The substrate is heated at a temperature selected based on the solvent boiling point, melting temperatures of the precursors, the decomposition temperatures of the precursors, and the temperature at which lithium sublimation occurs. Lithium sublimation occurs at temperatures of about 690° C. and higher, and results in lithium loss from the thin film. In one embodiment, the temperature of the substrate, the decomposition temperature of the precursors, and the solvent boiling point are within a narrow temperature range (e.g., a 100° C. range, a 50° C. range, a 25° C. range, a 10° C. range, or a 5° C. range). This embodiment may be preferable for depositing a more uniform film composition with lower amounts of impurities. The substrate is heated at a temperature of about 80° C. to about 450° C. In one embodiment, the substrate is heated at about 100° C. to about 400° C. In another embodiment, the substrate is heated at about 180° C. to about 220° C. (e.g., about 180° C., 200° C., or 220° C.).

FIG. 1B shows the annealing process used to convert the as-deposited thin film 150a to the post-annealed film 160 at an elevated temperature following deposition. Annealing the thin film may decompose precursor compounds to form the desired lithium salt. Annealing may also decompose residual solvent present in the thin film. Annealing may also promote the formation of a desired salt phase in the film, promote the growth of crystalline grains in the film, and/or densify the film. FIG. 1B shows a tube furnace 190 used for the annealing process, but other forms of heating may be used. The thin film 150a may be annealed using a heating element. The heating element may be a convection heater, an infrared heater, a resistive heater, or a combination thereof. For example, the heating element may be a tube furnace. The thin film 150a may be annealed at a temperature such that the thing film 150a has a temperature of about 100° C. to about 800° C. Preferably, during annealing, the thin film 150a has a temperature of about 350° C. to about 550° C. (e.g., 350° C., 400° C., 450° C., 500° C., or 550° C.). The heating element thermally coupled to the thin film 150a may be heated to the desired temperature using a selected ramp rate of about 5° C. per minute (min) to about 25° C. per min (e.g., 5° C. per min, 10° C. per min, 15° C. per min, 20° C. per min, or 25° C. per min). The thin film may be annealed at the selected temperature for a period of time of about 5 min to about 4 hours (hr) (e.g., 5 min, 10 min, 15 min, 30 min, 1 hr, 2 hr, 3 hr, or 4 hr). The film may be annealed in air, synthetic air (about 20% O₂ and 80% N₂), O₂, or an inert gas (e.g., argon or nitrogen).

In some cases, the thin film 150a is not post-annealed to achieve the desired material. Post-annealing is not necessary when the precursor solution 120 directly deposits the lithium salt desired. For example, a precursor solution 120 including lithium hypochlorite solution is deposited directly to form a solid lithium hypochlorite thin film.

The as-deposited or post-annealed metal salt thin film has a metal ion in the metal salt that is at least one of an alkali metal ion or an alkaline earth metal ion. The metal salt film may be a lithium metal salt film. The lithium metal salt film may include lithium nitrate, lithium nitride, lithium sulfate, lithium phosphate, lithium fluoride, lithium chloride, lithium hydroxide, lithium perchlorate, lithium azide, or a combination thereof. The substrate 140b is a solid material that is substantially stable at temperatures up to at least about 800° C. It may include magnesium oxide, aluminum oxide, silicon dioxide, indium tin oxide (ITO), zinc oxide, indium tin zinc oxide (ITZO), silicon carbide, titanium, nickel, stainless steel, or a combination thereof.

The thickness of the lithium salt film can be selected by changing deposition parameters. These parameters include the spray rate, spray time, substrate temperature during spray deposition, post-annealing temperature, and post-annealing time. Increasing the total volume of spray solution, which is dependent on the spray rate and spray time, may increase the film thickness. Increasing post-annealing temperature and post-annealing time may decrease the film thickness. The lithium salt film has a thickness of about 400 nm to about 50 μm. Preferably, the lithium salt film has a thickness of about 1 μm to about 10 μm (e.g., 1 μm, 2 μm, 3 μm, 4 μm, 5 μm, 6 μm, 7 μm, 8 μm, 9 μm, or 10 μm).

The phase and crystallinity of the lithium salt film can also be selected by changing deposition parameters. These parameters include post-annealing temperature, post-annealing ramp rate up to the temperature selected for post-annealing, ramp rate down to cool the lithium salt film, the type of carrier gas used for post-annealing, and post-annealing time. The lithium salt film may be amorphous or crystalline. Increasing the annealing temperature and/or annealing time may increase the lithium salt film's crystallinity.

The surface roughness and morphology of the lithium salt film can also be selected by changing deposition parameters. These parameters include spray rate, substrate temperature during spray deposition, precursor solution solvent, post-annealing temperature, post-annealing time, and post-annealing temperature ramp rate. Increasing the post-annealing temperature and/or post-annealing time may increase the surface roughness of the lithium salt film.

In another embodiment, the metal salt film may be a composite salt film. The composite salt thin film may include at least one lithium salt (e.g., any of the lithium salts described above) and at least one other metal salt. The ratio of lithium salt to metal salt may be about 4:1 to about 1:4 (e.g., 4:1, 2:1, 1:1, 1:2, or 1:4). The composite salt film can have the same thickness, phase, crystallinity, and surface roughness as the lithium salt film described above.

The other metal salt in the composite thin film may include zinc nitrate, zinc nitride, zinc sulfate, zinc phosphate, zinc fluoride, zinc chloride, zinc hydroxide, zinc perchlorate, zinc azide, calcium nitrate, calcium nitride, calcium sulfate, calcium phosphate, calcium fluoride, calcium chloride, calcium hydroxide, calcium perchlorate, calcium azide, potassium nitrate, potassium nitride, potassium sulfate, potassium phosphate, potassium fluoride, potassium chloride, potassium hydroxide, potassium perchlorate, potassium azide, sodium nitrate, sodium nitride, sodium sulfate, sodium phosphate, sodium fluoride, sodium chloride, sodium hydroxide, sodium perchlorate, sodium azide, bismuth nitrate, bismuth nitride, bismuth sulfate, bismuth phosphate, bismuth fluoride, bismuth chloride, bismuth hydroxide, bismuth perchlorate, bismuth azide, cerium nitrate, cerium nitride, cerium sulfate, cerium phosphate, cerium fluoride, cerium chloride, cerium hydroxide, cerium perchlorate, cerium azide, iron nitrate, iron nitride, iron sulfate, iron phosphate, iron fluoride, iron chloride, iron hydroxide, iron perchlorate, iron azide, zirconium nitrate, zirconium nitride, zirconium sulfate, zirconium phosphate, zirconium fluoride, zirconium chloride, zirconium hydroxide, zirconium perchlorate, zirconium azide, yttrium nitrate, yttrium nitride, yttrium sulfate, yttrium phosphate, yttrium fluoride, yttrium chloride, yttrium hydroxide, yttrium perchlorate, yttrium azide, lanthanum nitrate, lanthanum nitride, lanthanum sulfate, lanthanum phosphate, lanthanum fluoride, lanthanum chloride, lanthanum hydroxide, lanthanum perchlorate, lanthanum azide, tantalum nitrate, tantalum nitride, tantalum sulfate, tantalum phosphate, tantalum fluoride, tantalum chloride, tantalum hydroxide, tantalum perchlorate, tantalum azide, barium nitrate, barium nitride, barium sulfate, barium phosphate, barium fluoride, barium chloride, barium hydroxide, barium perchlorate, barium azide, strontium nitrate, strontium nitride, strontium sulfate, strontium phosphate, strontium fluoride, strontium chloride, strontium hydroxide, strontium perchlorate, strontium azide, indium nitrate, indium nitride, indium sulfate, indium phosphate, indium fluoride, indium chloride, indium hydroxide, indium perchlorate, indium azide, magnesium nitrate, magnesium nitride, magnesium sulfate, magnesium phosphate, magnesium fluoride, magnesium chloride, magnesium hydroxide, magnesium perchlorate, magnesium azide, scandium nitrate, scandium nitride, scandium sulfate, scandium phosphate, scandium fluoride, scandium chloride, scandium hydroxide, scandium perchlorate, scandium azide, aluminum nitrate, aluminum nitride, aluminum sulfate, aluminum phosphate, aluminum fluoride, aluminum chloride, aluminum hydroxide, aluminum perchlorate, aluminum azide, lutetium nitrate, lutetium nitride, lutetium sulfate, lutetium phosphate, lutetium fluoride, lutetium chloride, lutetium hydroxide, lutetium perchlorate, lutetium azide, niobium nitrate, niobium nitride, niobium sulfate, niobium phosphate, niobium fluoride, niobium chloride, niobium hydroxide, niobium perchlorate, niobium azide, antimony nitrate, antimony nitride, antimony sulfate, antimony phosphate, antimony fluoride, antimony chloride, antimony hydroxide, antimony perchlorate, antimony azide, tin nitrate, tin nitride, tin sulfate, tin phosphate, tin fluoride, tin chloride, tin hydroxide, tin perchlorate, tin azide, hafnium nitrate, hafnium nitride, hafnium sulfate, hafnium phosphate, hafnium fluoride, hafnium chloride, hafnium hydroxide, hafnium perchlorate, hafnium azide, tungsten nitrate, tungsten nitride, tungsten sulfate, tungsten phosphate, tungsten fluoride, tungsten chloride, tungsten hydroxide, tungsten perchlorate, tungsten azide, gallium nitrate, gallium nitride, gallium sulfate, gallium phosphate, gallium fluoride, gallium chloride, gallium hydroxide, gallium perchlorate, gallium azide,

strontium nitrate, strontium nitride, strontium sulfate, strontium phosphate, strontium fluoride, strontium chloride, strontium hydroxide, strontium perchlorate, strontium azide, or a combination thereof. The other metal salt in the composite thin film may include other ions in the alkali metal family and the Lanthanide series. The other metal salt in the composite thin film may include a metalloid ion including silicon, germanium, selenium, or a combination thereof.

FIG. 1C shows part of a spray pyrolysis system 102 disposed inside of an enclosure 180 providing a substantially inert atmosphere. In some embodiments, the spray pyrolysis system 102 is used to deposit the thin film 150c with little to no oxygen gas present. This reduces or prevents the formation of oxygen impurities in the thin film 150c. The system 102 includes a spray nozzle 110c (e.g., an atomizer) that is fluidically coupled to a precursor solution 120. The spray nozzle 110c produces a spray mist 130c inside of the enclosure 180. The spray mist 130c includes droplets of precursor solution 120c. The spray mist 130c is directed towards a substrate 140c, where the thin film 150c is formed. The system 102 may use a carrier gas to create the spray mist 130c. The carrier gas is an inert gas (e.g., argon, nitrogen, or sulfur hexafluoride). The substrate 140c is thermally coupled to a heating element 142c that heats the substrate 140c. The enclosure 180 includes an inlet 182 providing an inert gas (e.g., argon, nitrogen, or sulfur hexafluoride) to the enclosure 180. In some cases, the enclosure 180 also includes an outlet 184 providing a gas outlet for pressure regulation and efficient purging of the enclosure 180.

FIG. 1D shows a lithium salt film 160d disposed on a substrate 140d, with a capping layer 170d disposed on the surface of the lithium salt film 160d. The spray pyrolysis system 102 may further include components to deposit the capping layer 170d on top of the thin film. The capping layer reduces or prevents the formation of oxygen impurities in the thin film 160d.

### Applications

Lithium salt thin films deposited using the systems and methods described above are potentially useful in many applications. Many lithium salts are electrically resistive and ionically conductive, making them particularly useful in electrochemical applications. For example, lithium salts thin films are used as solid electrolytes in solid-state lithium batteries. Solid electrolytes have the same role as traditional liquid electrolytes; they transport lithium ions between the cathode and the anode. Inorganic solid electrolytes are crystalline or glass materials that conduct lithium ions by diffusion through the crystal lattice.

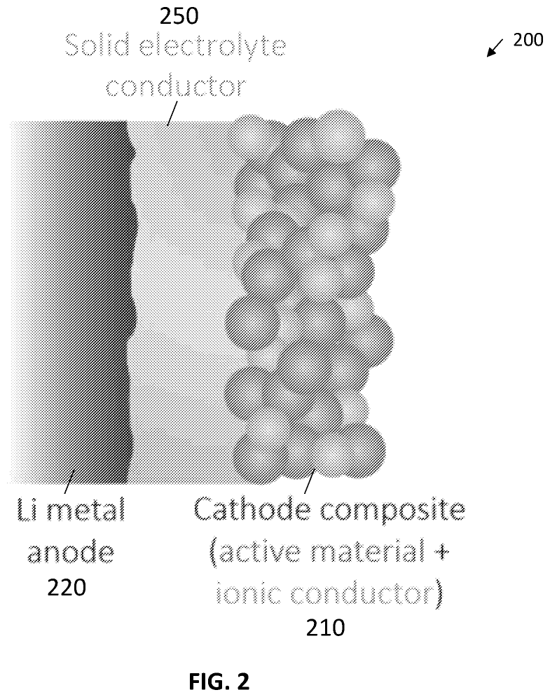

FIG. 2 shows a partial cross-section of a solid-state lithium battery 200. The solid-state battery 200 includes a composite cathode 210, a lithium metal anode 220, and a solid electrolyte 250. The composite cathode 210 includes an active material and an ionic conductor. The solid electrolyte 250 is disposed between the composite cathode 210 and the lithium metal anode 220. The solid electrolyte 250 forms interfaces with the cathode 210 and the anode 220 so that lithium ions are conducted between the cathode 210 and the anode 220 through the solid electrolyte 250.

The solid electrolyte 250 may include a single component amorphous lithium salt, or a composite that includes at least one amorphous lithium salt deposited using the systems and methods described above. Lithium salts that may be included in the solid electrolyte 250 include lithium nitride, lithium hypochlorite, and lithium halides (e.g., lithium fluoride, lithium chloride, and lithium bromide). For use as solid electrolytes, lithium salt films are deposited using parameters chosen to deposit a film with a desired surface roughness.

As another example, lithium salt thin films deposited using the systems and methods described above are used as sensing electrodes and solid electrolytes in potentiometric electrochemical gas sensors. These lithium-based potentiometric sensors are less susceptible to corrosion and more selective at low operating temperatures when compared with conventional semiconducting metal oxide sensors. In a solid-state electrochemical gas sensor, a chemical gas species reacts at the electrode/ion-conductor interface where electrical charges are exchanged, resulting in an electrical signal that is related to the concentration or partial pressure of the gas species. These potentiometric sensors can detect complex gas species, including CO₂, NO₂, and SO₂.

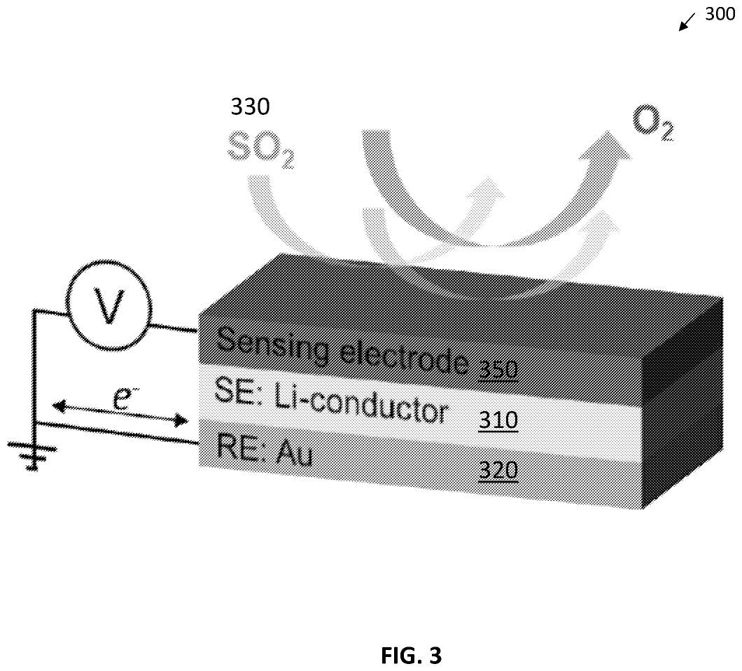

FIG. 3 shows a schematic of a potentiometric gas sensor 300. The potentiometric gas sensor 300 includes a reference/counter electrode 320, a solid electrolyte 310 disposed on the reference/counter electrode 320 and a sensing electrode 350 disposed on the solid electrolyte 310. The gas sensor 300 detects SO₂, but other complex gas species (e.g., CO₂, and NO₂) may be detected using a gas sensor with a similar configuration. The sensing electrode 350 includes a thin film that is deposited using the systems and methods described above. As an example, the thin film may be lithium sulfate or a composite including lithium sulfate and calcium sulfate. For use as sensing electrode, lithium salt films are deposited using parameters chosen to increase surface roughness in order to increase the available surface area for gas detection. For example, a higher ramp rate during the annealing process may be used to increase the surface roughness of the film (e.g., increasing the ramp rate from 10° C. per minute to 20° C. per minute).

### Examples

Lithium sulfate films and composite films including lithium sulfate and calcium sulfate were deposited on magnesium oxide substrates. Lithium acetate, ammonium sulfate, and calcium acetate were used as precursors in the precursor solutions. The solvent in the precursor solution was water, in which all three precursor compounds are soluble. To deposit a lithium sulfate film, the precursor solution included aqueous lithium acetate in a concentration of about 0.03 M (representing 50% over-lithiation) and aqueous ammonium sulfate in a concentration of about 0.01M. To deposit the composite film, the precursor solution included aqueous lithium acetate in a concentration of about 0.015 M (representing 50% over-lithiation), aqueous calcium acetate in a concentration of about 0.005 M (representing a 1:1 ratio of Li:Ca in the resulting composite film), and aqueous ammonium sulfate in a concentration of about 0.01 M.

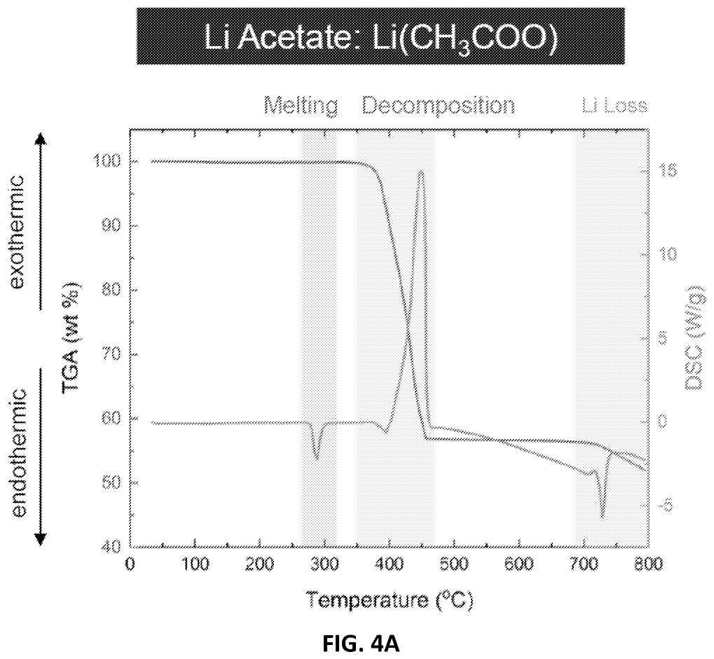

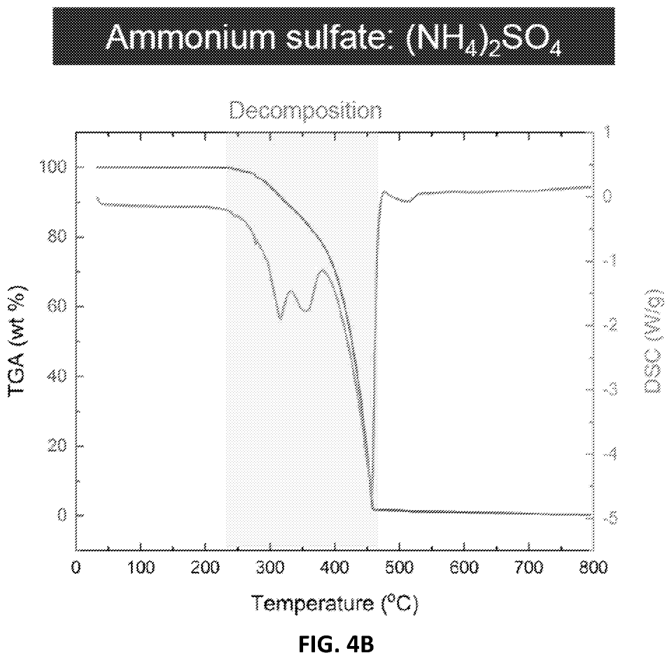

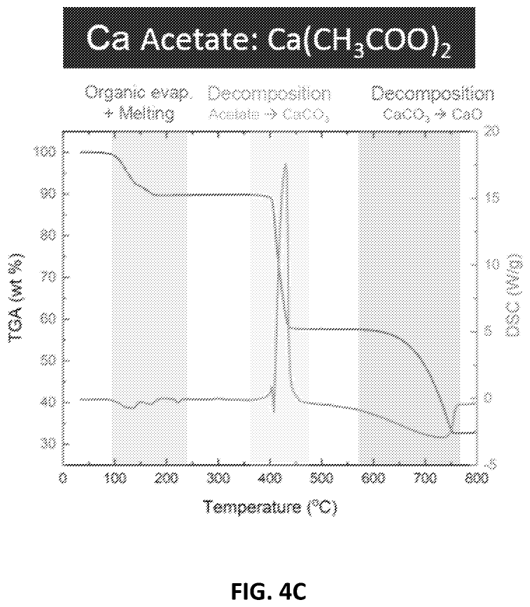

FIGS. 4A-4C show thermogravimetric analysis (TGA) and differential scanning calorimetry (DSC) results for the precursors lithium acetate, ammonium sulfate, and calcium acetate, respectively. The results shown in FIGS. 4A-4C indicate the melting temperature range, the decomposition temperature range, and the temperature of the onset of lithium loss. In FIG. 4A, lithium acetate decomposition began at a temperature of about 300° C. and lithium loss began at a temperature of about 690° C. In FIG. 4B, ammonium sulfate decomposition began at a temperature of about 230° C. In FIG. 4C, calcium acetate had a first decomposition from calcium acetate to calcium carbonate starting at about 355° C. and a second decomposition from calcium carbonate to calcium oxide starting at about 575° C.

Along with the solvent boiling point (here, 100° C.), the precursor decomposition temperature was considered when selecting the temperature of the hotplate that heats the substrate during deposition. The following thin films were deposited with a hotplate temperature of about 200° C.±10° C. The hotplate temperature was set between the precursors' decomposition temperatures (>300° C.) and the solvent boiling point (100° C.).

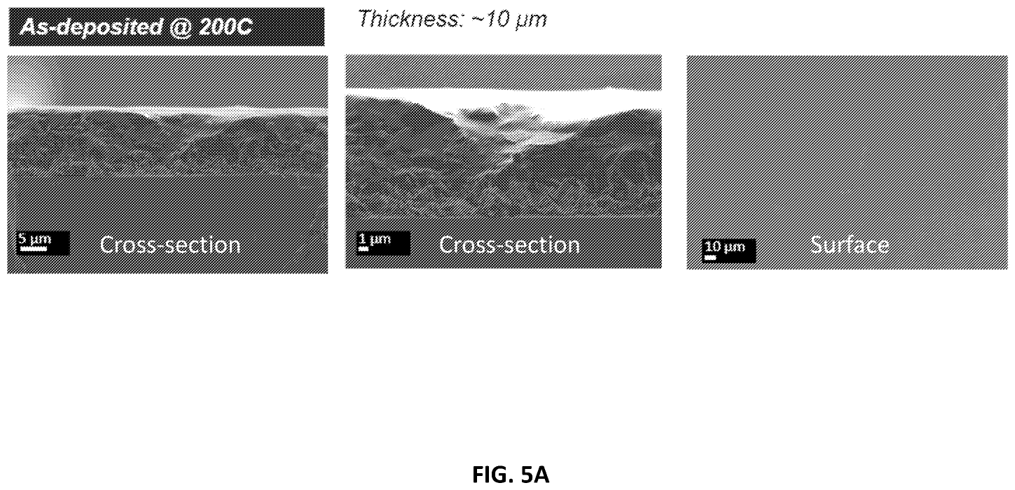

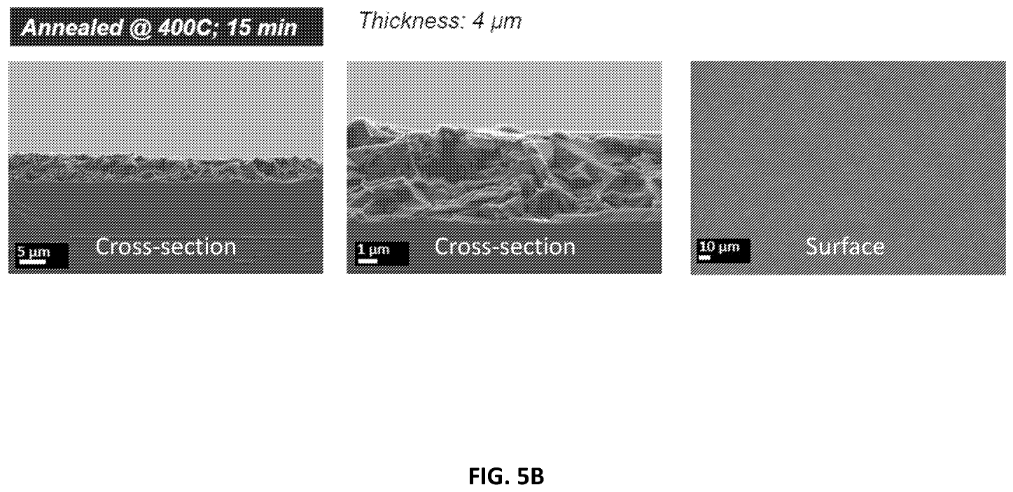

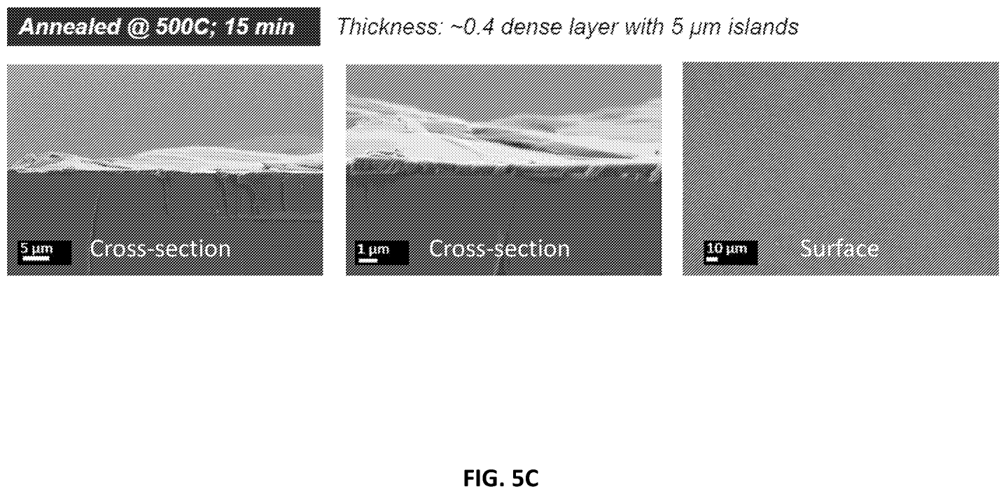

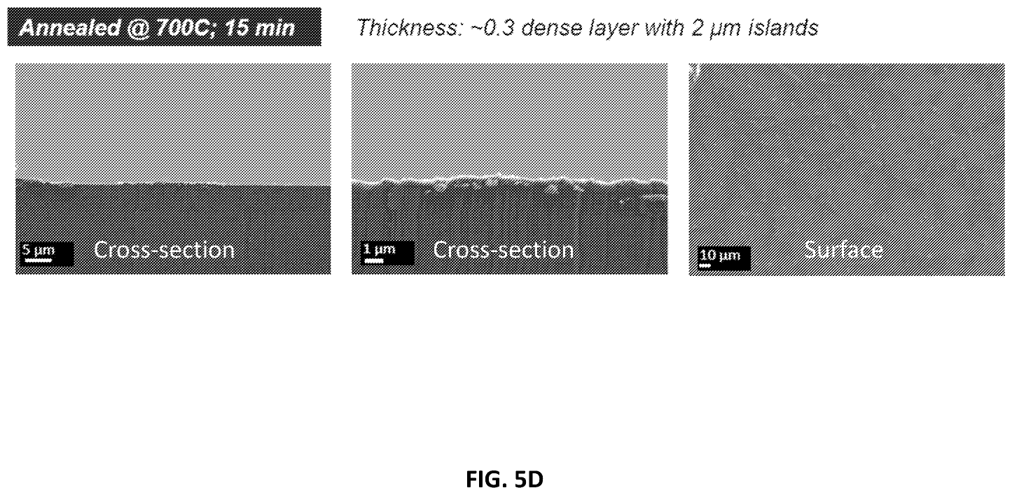

FIGS. 5A-5D show lithium sulfate films deposited using spray pyrolysis at a spray rate of 5 mL per hr. for 3 hrs., resulting in 15 mL of precursor solution sprayed. FIG. 5A shows a lithium sulfate film as deposited at 200° C. having a thickness of about 10 μm. FIG. 5B shows a lithium sulfate film after annealing at 400° C. for 15 mins of isothermal time in a tube furnace with a ramp rate of 10° C. per min under synthetic air. After annealing at 400° C., the lithium sulfate film has a thickness of about 4 μm. FIG. 5C shows a lithium sulfate film after annealing at 500° C. having a thickness of about 0.4 μm. This film also had islands of the same material disposed at the surface of the film, having a thickness of about 6 μm. FIG. 5D shows a lithium sulfate film after annealing at 700° C. having a thickness of about 0.3 μm with 2 μm islands disposed at the surface of the film.

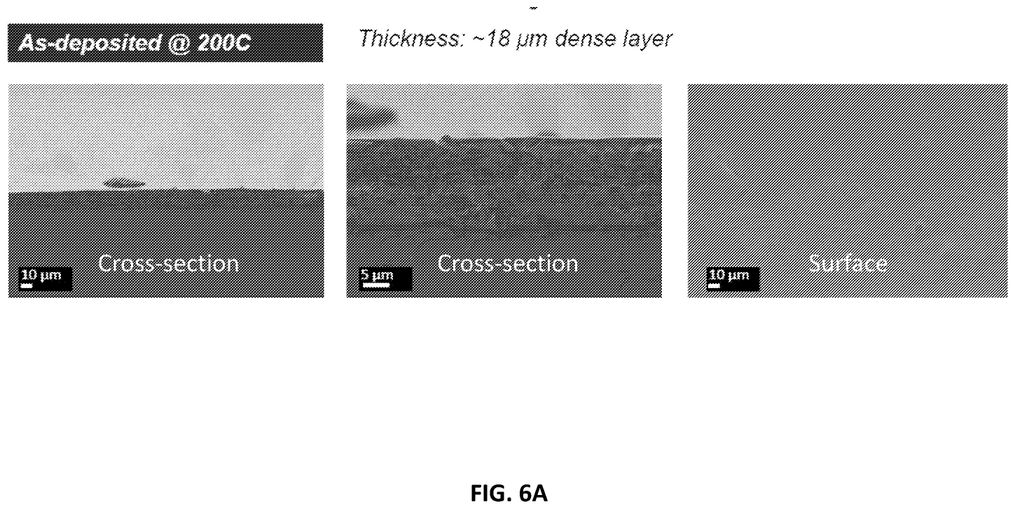

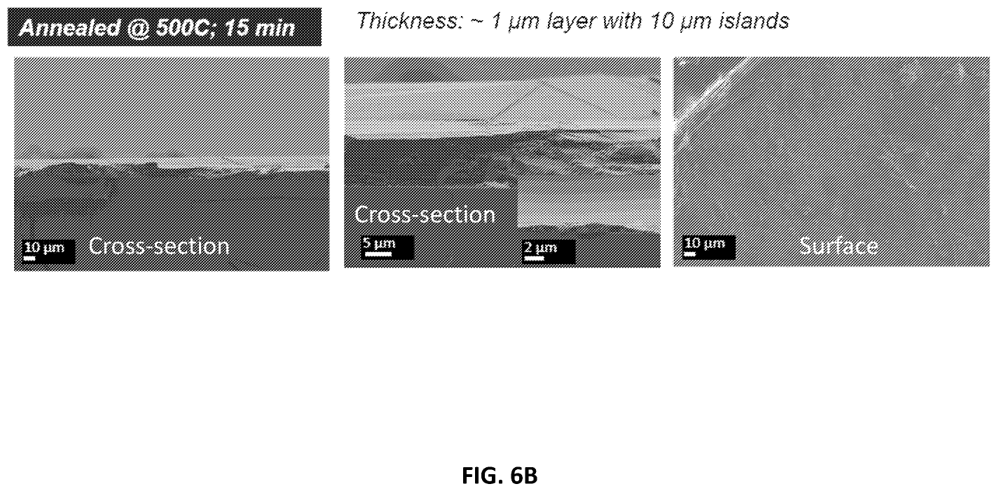

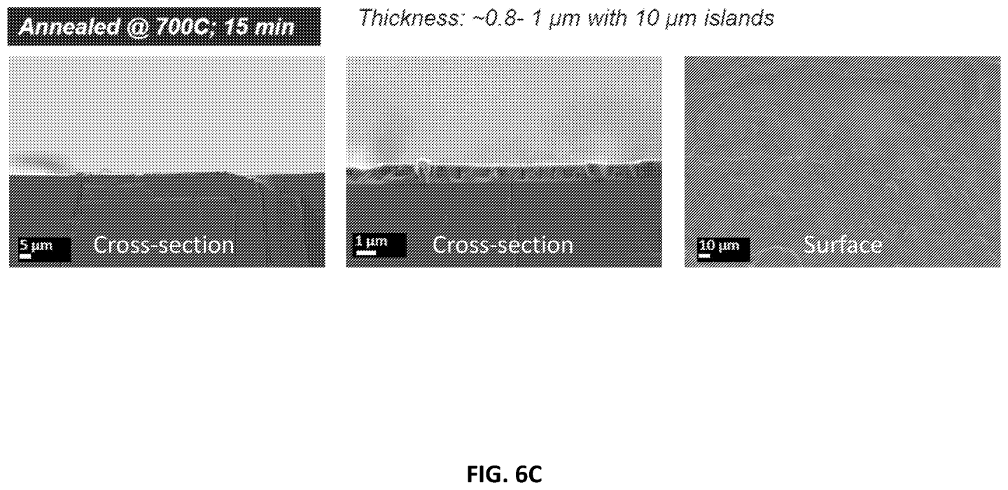

FIGS. 6A-6C show lithium sulfate films deposited using spray pyrolysis at a spray rate of 10 mL per hr. for 2 hrs., resulting in 20 mL of precursor solution sprayed. FIG. 6A shows a lithium sulfate film as deposited having a thickness of about 18 μm. FIG. 6B shows a lithium sulfate film after annealing at 500° C. for 15 mins having a thickness of about 1 μm with 10 μm islands. FIG. 6C shows a lithium sulfate film after annealing at 700° C. for 15 mins having a thickness of about 0.8 μm to about 1 μm with 10 μm islands.

The results in FIGS. 5A-6C indicated that the thickness of the lithium sulfate film decreases with increasing annealing temperature. As indicated in the Raman spectra below, film thickness may be reduced by the removal of precursor residues from the film. Film thickness reduction may result from film densification and increase film crystallinity. At 700° C., the reduction in film thickness may also result from some lithium loss. The results also indicated that the lithium sulfate phase started to form even in the as-deposited film. However, precursors in the films were not fully decomposed until annealed at elevated temperatures, where the film had a temperature of about 500° C. or higher. The results also indicated that increasing the annealing temperature increased the crystallinity of the film and the surface roughness of the film.

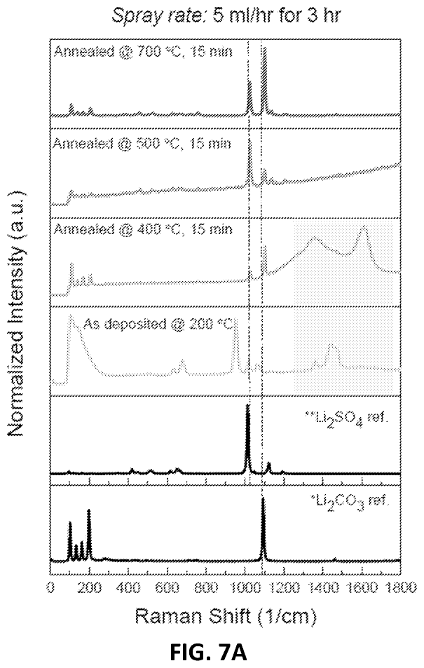

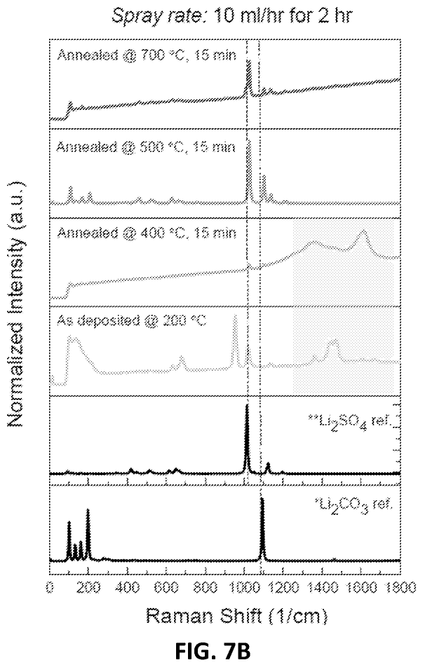

FIGS. 7A and 7B show Raman spectra for the lithium sulfate films shown in FIGS. 5A-5D and FIGS. 6A-6C, respectively. The Raman spectra are compared to reference Raman spectra for lithium sulfate and lithium carbonate. The results indicate that lithium sulfate is present in the films as deposited and in all the films after post-annealing. Spectra for the films as deposited also had other large peaks that indicated the presence of precursor residues in the films. The precursor residue signatures were present to a lesser extent in the films post-annealed at 400° C., indicating the presence of a small amount of precursor residue in these films. The precursor residue signatures were substantially absent from the films post-annealed at 500° C. and 700° C. Many of the spectra had peaks corresponding to lithium carbonate, indicating that a small amount of lithium sulfate reacted with carbon dioxide in the air during or after the annealing step to form lithium carbonate. Lithium carbonate impurities can be prevented by forming a capping layer on the lithium sulfate film after post-annealing. Comparing the results in FIGS. 7A and 7B indicate successful deposition of lithium sulfate using a spray rate of 5 mL per hr and 10 mL per hr.

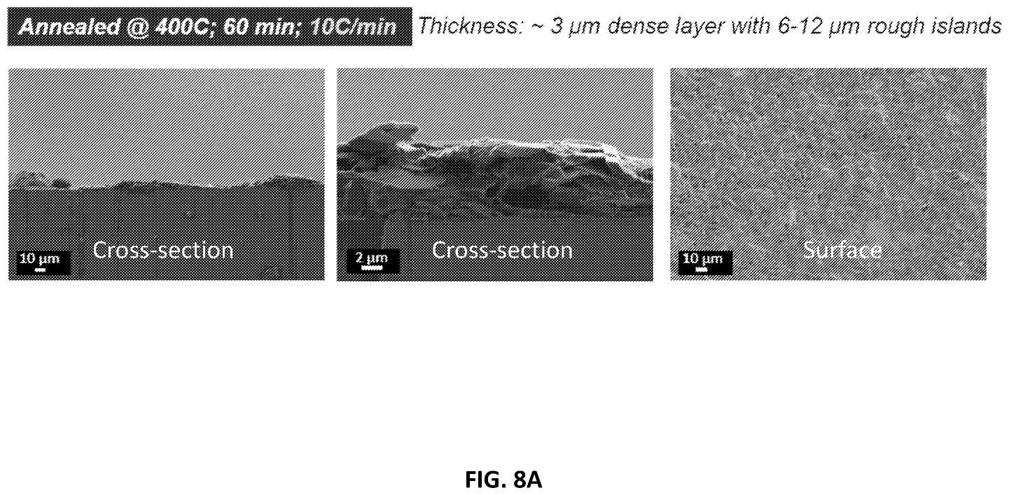

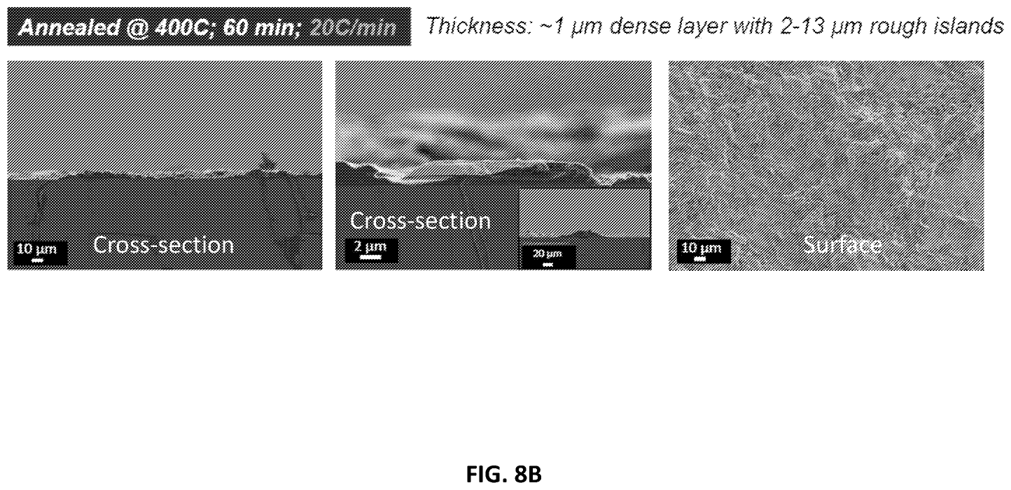

FIGS. 8A and 8B show lithium sulfate films after annealing at 400° C. for 60 minutes using a heating ramp rate of 10° C. per min and 20° C. per min, respectively. The lithium sulfate film annealed with a 10° C. per min ramp rate had a thickness of 3 μm with 6 μm to 12 μm islands on the surface of the film. The lithium sulfate film annealed with a 20° C. per min ramp rate had a thickness of about 1 μm with 2 μm to 13 μm islands on the surface. The results indicate that faster ramp rates result in films with more morphology variability and surface roughness.

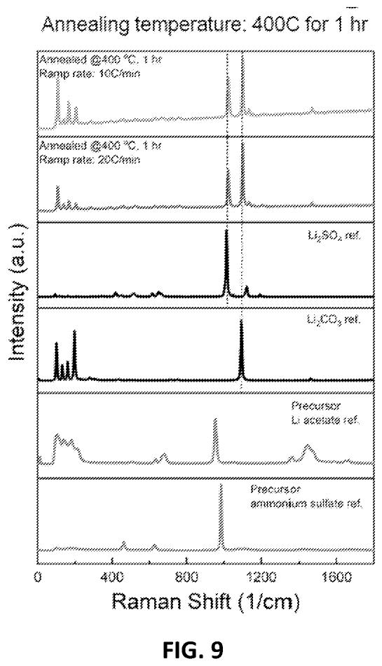

FIG. 9 shows Raman spectra for the lithium sulfate films shown in FIGS. 8A and 8B. Both films had prominent peaks indicating the presence of lithium sulfate. After annealing at 400° C. for 1 hr, the lithium sulfate films did not have precursor residues present in their Raman spectra. The results indicated that both ramp rates result in lithium sulfate films, and that ramp rate can be selected to change the morphology of the resulting film.

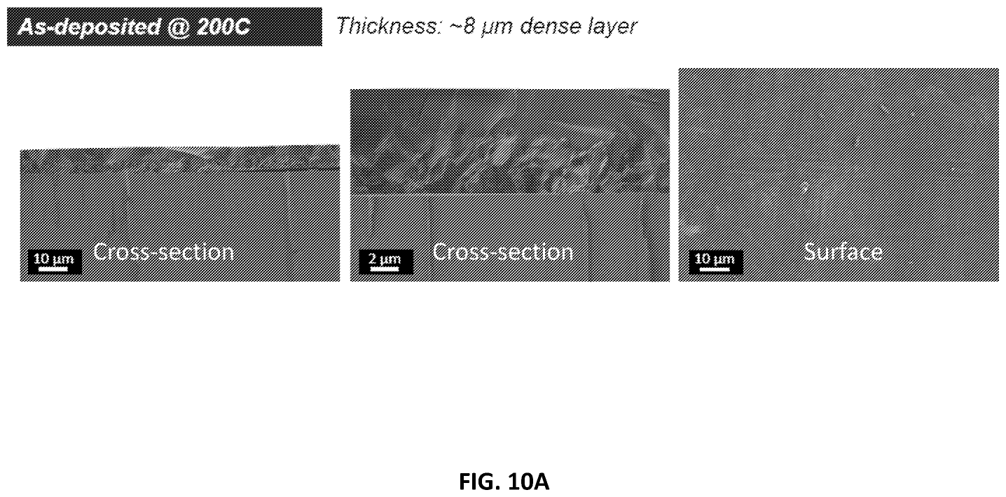

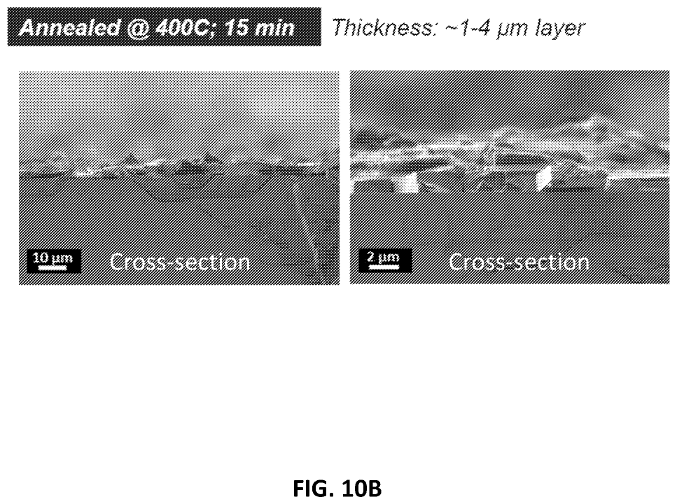

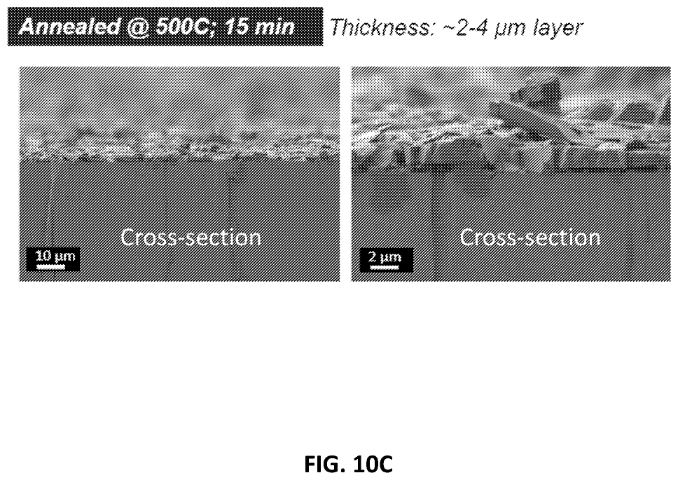

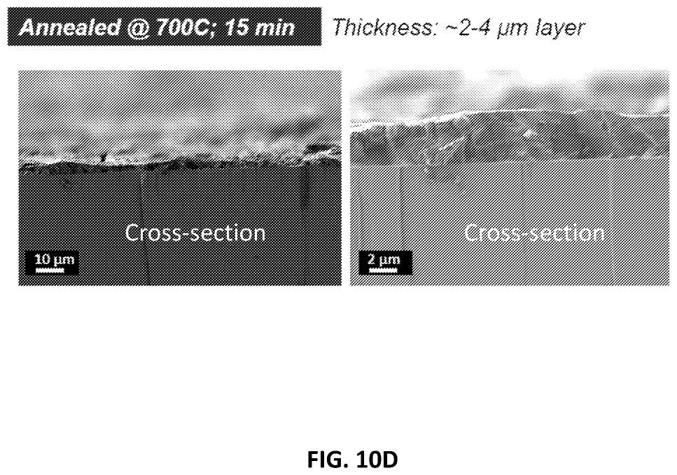

FIGS. 10A-D show composite films of lithium sulfate and calcium sulfate deposited in a 1:1 ratio. The composite films were deposited using spray pyrolysis at a spray rate of 10 mL per hr. for 1 hr., resulting in 10 mL of precursor solution sprayed. FIG. 10A shows the composite film as deposited having a thickness of about 8 μm. FIG. 10B shows the composite film after annealing at 400° C. for 15 min having a thickness of about 1 μm to about 4 μm with islands disposed at the film surface. FIG. 10C shows the composite film after annealing at 500° C. for 15 min having a thickness of about 2 μm to about 4 μm with islands disposed at the film surface. FIG. 10D shows the composite film after annealing at 700° C. for 15 min having a thickness of about 2 μm to about 4 μm with islands disposed at the film surface. These results indicate that post-annealing causes a decrease in film thickness, an increase in surface roughness, and an increase in variability of film morphology.

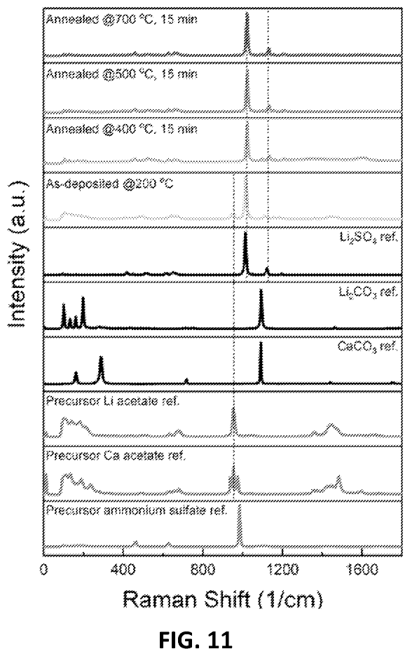

FIG. 11 shows Raman spectra for the composite films shown in FIGS. 10A-10D. The spectra are compared to reference spectra for lithium sulfate, lithium carbonate, calcium carbonate, precursor lithium acetate, precursor calcium acetate, and precursor ammonium sulfate. All the films, including the film as deposited, had prominent peaks indicating the presence of lithium sulfate. The film as deposited had weak peaks indicating the presence of residual precursors, including lithium acetate and calcium acetate. These precursor peaks were substantially absent from the post-annealed films, indicating that precursor residues were not present in these films.

A lithium hypochlorite thin film was deposited on a magnesium oxide substrate using spray pyrolysis. The precursor solution was lithium hypochlorite salt dissolved in methanol at a concentration of about 0.02 M. The hot plate was heated at a temperature of about 100° C.±10° C. during deposition. The spray rate was 10 mL per hour and the spray time was 1 hour, so that the total volume sprayed was 10 mL.

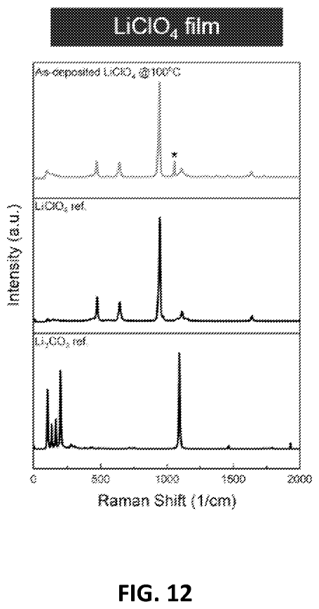

FIG. 12 shows a Raman spectrum of the as-deposited lithium hypochlorite film. The lithium hypochlorite film was compared to a reference lithium hypochlorite spectrum and a reference lithium carbonate spectrum. The Raman spectrum shows a prominent peak indicating lithium hypochlorite. Because the film was directly deposited, a post-annealing step was not used. In other words, precursor decomposition was not used to form the film.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for making a lithium salt film, the method comprising:
heating a substrate;
while heating, spraying a mixture onto the substrate to form a precursor film, the mixture comprising:
a first precursor comprising a lithium ion;
a second precursor comprising an anion; and
a solvent; and
annealing the precursor film to form the lithium salt film having a thickness of about 400 nm to about 100 μm;
wherein:
the lithium salt film comprises the lithium ion and the anion; and
while annealing, the precursor film has a temperature of 100° C. to 800° C.

2. The method of claim 1, wherein:
the lithium ion is dissolved in the solvent; and
a concentration of the lithium ion in the solvent is a stoichiometric excess greater than a stoichiometric amount of the lithium ion in the lithium salt film.

3. The method of claim 2, wherein the stoichiometric excess is about 5% to about 300%.

4. The method of claim 1, wherein the solvent comprises at least one of water, an alcohol, an ester, a carbonate, or a ketone.

5. The method of claim 1, wherein the first precursor comprises at least one of lithium acetate, lithium nitrate, lithium hydroxide, or lithium azide.

6. The method of claim 1, wherein the second precursor comprises at least one of a sulfate ion, a hydroxide ion, nitrate ion, a nitride ion, a phosphate ion, a fluoride ion, a chloride ion, a bromide ion, a perchlorate ion, or an azide ion.

7. The method of claim 6, wherein the second precursor comprises ammonium sulfate.

8. The method of claim 1, wherein the lithium salt film comprises at least one of lithium nitrate, lithium nitride, lithium sulfate, lithium phosphate, lithium fluoride, lithium chloride, lithium bromide, lithium hydroxide, lithium perchlorate, or lithium azide.

9. The method of claim 1, wherein the substrate is a solid substrate comprising at least one of a metal, a metal oxide, a metal nitride, a metal alloy, a borosilicate, carbon, silicon, silicon dioxide, or a polyimide.

10. The method of claim 1, further comprising depositing a capping layer on a surface of the lithium salt film.

11. The method of claim 1, wherein:
the mixture further comprises a third precursor comprising a second metal ion; and
the lithium salt film further comprises the second metal ion.

12. The method of claim 11, wherein the second metal ion comprises at least one of a zinc ion, a calcium ion, a potassium ion, a sodium ion, a bismuth ion, a cerium ion, a zirconium ion, an iron ion, a yttrium ion, a lanthanum ion, a tantalum ion, a beryllium ion, a barium ion, a strontium ion, an indium ion, a magnesium ion, an aluminum ion, a lutetium ion, a niobium ion, an antimony ion, a tin ion, a hafnium ion, a tungsten ion, a silicon ion, a selenium ion, a gallium ion, a germanium ion, another alkali metal ion, or another lanthanide series metal ion.

13. The method of claim 11, wherein the first precursor comprises lithium sulfate and the third precursor comprises calcium sulfate.

14. The method of claim 13, wherein the lithium sulfate and the calcium sulfate are in a mole ratio of about one to one.

15. An electrochemical gas sensor comprising a sensing electrode comprising the lithium salt film of claim 1.

16. A method for making a lithium sulfate film, the method comprising:
spraying a mixture onto a substrate having a temperature of about 180° C. to 220° C. to form a precursor film, the mixture comprising:
lithium acetate,
ammonium sulfate, and
water; and
annealing the precursor film to form the lithium sulfate film having a thickness of about 1 μm to about 10 μm;
wherein, while annealing, the precursor film has a temperature of 400° C. to 700° C.

17. An electrochemical sulfur dioxide gas sensor comprising a sensing electrode comprising the lithium sulfate film of claim 16.

18. A lithium salt film deposition system comprising:
a spray pyrolysis apparatus comprising:
a precursor solution comprising lithium acetate and ammonium sulfate;
a spray nozzle to create a spray mist of the precursor solution;
a substrate positioned so as to be contacted by at least a portion of the spray mist; and
a first heating source configured to heat the substrate; and
a post-annealing apparatus comprising:
a chamber;
a second heating source thermally coupled to the chamber and configured to heat the substrate so that the substrate has a temperature of 100° C. to 800° C.

19. The lithium salt deposition system of claim 18, wherein the first heating source is configured to heat the substrate so that the substrate has a temperature of about 100° C. to about 400° C.

20. The lithium salt deposition system of claim 18, wherein the post-annealing apparatus is a tube furnace further comprising an inert gas source fluidically coupled to the chamber.
